(19) United States
(12) Patent Application Publication
(10) Pub. No.: US 2018/0206835 A1
(43) Pub. Date: Jul. 26, 2018

(54) INJECTOR-FREE IRIS EXPANSION RING FOR CATARACT SURGERY

(71) Applicant: Sérgio Félix CANABRAVA, Funcionários, Belo Horizonte (BR)

(72) Inventor: Sérgio Félix CANABRAVA, Funcionários, Belo Horizonte (BR)

(21) Appl. No.: 15/745,570

(22) PCT Filed: Jul. 31, 2015
(86) PCT No.: PCT/BR2015/000119

§ 371 Date: Jan. 17, 2018

(51) Int. Cl. A61B 17/02

(52) U.S. Cl. CPC A61B 17/0231; A61F 9/007; A61B 17/0293

(57) ABSTRACT

The present patent request refers to a utility model for an iris expansion ring which conjugates the functions of iris expansion and pupil dilation on patients that will be subjected to ophthalmic surgeries, and also permits implantation e withdrawal with a single incision. Handling of the present device spares accessory incisions to the ocular globe as well as the use of injector for its insertion, also displaying an alternated indents structure and low thickness that renders the whole implantation process easier.

The present patent request refers to a utility model for an iris expansion ring which conjugates the functions of iris expansion and pupil dilation on patients that will be subjected to ophthalmic surgeries, and also permits implantation e withdrawal with a single incision. Handling of the present device spares accessory incisions to the ocular globe as well as the use of injector for its insertion, also displaying an alternated indents structure and low thickness that renders the whole implantation process easier.

Eye illnesses affect a considerable proportion of the world population. Advances in technology and ophthalmic medicine have made it possible to prevent, cure and treat some problems affecting the eye which used to be considered untreatable a few years ago. Most common ocular diseases include myopia, astigmatism, hypermetropia, glaucoma and cataract.

Cataract consists of the total or partial opacity in the crystalline lens, the natural lens in the ocular globe, which is responsible for focalizing eye sight either for near or far sight. It is the progressive diminishing of vision, congenital or acquired (this being the most frequent form of the disease). It affects about a half (46.2%) of world population over sixty five years old. It is estimated that around 160 million people in the world are affected by this disease, which is considered the major cause of avoidable blindness.

The curative treatment of cataract is surgical and consists of replacing the opaque crystalline lens for a prosthesis called intraocular lens (IOL).

As of now there are two main forms of dilating the pupil of patients which will undergo cataract surgery. The first one is using instruments called retractor hooks. Such instruments have the drawback of requiring four extra accessory incisions for its implantation during surgery. The other form is the use of an square-shaped expanding ring which requires an injector for implantation, and is currently protected by the copyright request BR 20200150056856. The requirement of an injector is a setback of this technology when compared to the one presented here, since it increases the cost of the product. Moreover, because it requires four support points on subject iris, it is impossible to use on patients presenting coloboma or sectorial loss of the iris due to trauma.

Intending to solve these inconveniences, the present expansion ring was developed, which can be implanted and withdrawn from subjects eye through the same incision used for extracting the cataract and with no need for an injector. Thus dispensing the need for four accessory incisions. The ring presents a predominantly circular shape, with an opening in one of its sections. Its internal diameter ranges from 5 to 8 mm, preferentially 6.3 mm, and its height ranges from 0.2 to 1.1 mm, preferentially 0.6 mm. It is composed of 3 to 10 alternated parts which fits to the eye iris, preferentially 7, consisting of larger parts in the superior section of the ring, followed by smaller parts in the inferior section, repeatedly. In which of these fitting parts there is an orifice for handling of the device inside the ocular globe.

Searching on data banks for patented devices the document U.S. Pat. No. 6,620,098 was found, which describes a device for pupil dilation or for keeping it opened, composed of an opening and a portion of body, having at least seven parts for fitting in the iris, and some of these parts may have internal orifices, and an arm, or hook, for fitting in the iris.

The present device technology differs from the one presented above by the fact that the parts for fitting in the iris are alternated, being one up-oriented and the other down-oriented, and due to this constructive layout, the ring's thickness is lower, and thus rendering it able to easily enter the ocular globe, bringing great advantages to the device's functionality, making implantation easier, since the iris connects like a wave to the ring.

Document U.S. Pat. No. 6,068,643 describes a device for dilating and keeping the pupil open which consists of a body displaying first and second terminations. The external peripheral edge of the body have an engaging formation, adapted to engage the peripheral interior edge of an iris to retain the pupil in an expanded status, and at least one positioning arm which extends out from one end of the body in such a manner that it keeps external to the eye.

Document US2014030550 describes an iris expansion device, which includes a body, unitary and non-metallic, of multiple segments, being expandable from a first status to a second status. This second status defines a bigger space than the first, with the body to be defined by a plurality of segments connected by living hinges. In another aspect, an iris expansion device is provided, which includes a multi-segmented body expandable from a first status to a second status of bigger grip.

Thus, none of the above mentioned documents compromise the present request for patenting of the technology here described, which conjugates the functions of pupil dilating, as well as allowing its implantation and withdrawal by a single incision.

The device conjugates the functions of expanding the iris and dilating the pupil, being an iris expanding ring to be used in ophthalmic surgeries for removal of cataract and insertion of intraocular lens. The ring is predominantly circular shaped, with an opening at its inferior section, sided by hook-shaped access points in both extremities; at least two superior structures for fitting, which docks on top of the iris, provided with an orifice for ring manipulation; at least one inferior structure for fitting in the iris edge, provided with an orifice for ring manipulation. The surface of the superior fitting structures which docks on top of the iris functions as ring support, to prevent decoupling and thus presents, preferably, a bigger area than the surface of the inferior structure. The fitting elements are positioned in the ring in an alternated manner, and the planes that contain these elements are preferably parallels and distanced, to create a socket for iris docking. The ring is composed of three to ten alternated iris-fitting elements, preferably seven.

The alternation between the superior and inferior fitting structures brings great advantages to the device's functionality, rendering it easier to implant, since the iris shall connect just like a wave on the ring. Moreover, it allows the ring to display a small thickness and thus facilitates its insertion in the eye.

The present patent request can be best understood through the attached figures, in which:

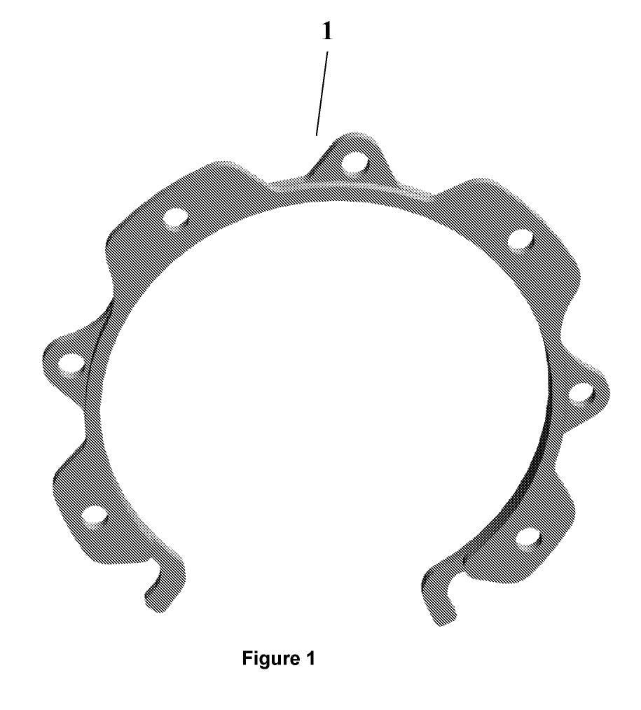

FIG. 1 represents the upper view of the ring.

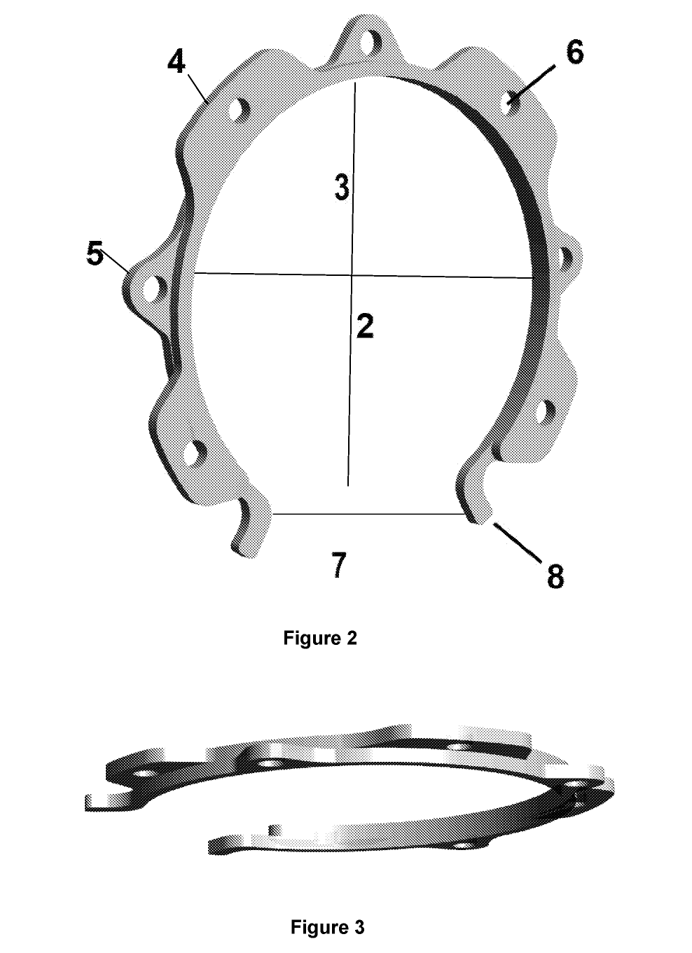

FIG. 2 represents a longitudinal view of the ring.

FIG. 3 represents a side view of the ring.

The device in its preferable form, but not limited, comprehends a ring (1) predominantly circular with a diameter (2) bigger in the center of the latitudinal section and height (3) higher in the longitudinal section, presenting an opening (7) in its inferior section, sided by support points (8) which are hook-shaped and situated in each of the inferior extremities; one to five, preferably three inferior structures (5) which fit to the edge of the iris, displaying triangular shape and an orifice (6) for ring manipulation (1), the superior structures (4) alternate along the circumference with the inferior structures (5), these being smaller than the superior ones (4), and the planes containing the superior structures (4) are parallels, the same happening for the inferior planes (5). These parts are distanced to create a socket for iris accommodation; the interior diameter of the ring ranges from 5 to 8 mm, preferably 6.3, and its height ranges from 0.2 to 1.1 mm, preferably 0.6 mm

Ring (1) implantation can be made through the same incision made for cataract withdrawal. Introduction can be started through any of the open parts (support points) of the ring (1).

The ring can be inserted to the ocular globe by simple tweezers commonly utilized in cataract surgery; once inserted inside, a hook must be used for manipulation of the cataract lens; the hook must be inserted in each orifice (6) connecting each structure, superior (4) and inferior (5), to the subject iris until the pupil is fully dilated by the ring; next, the surgery can be started and after the implantation of the intraocular lens, the lens manipulation hook is again used to disconnect each of the structures, superior (4) and inferior (5) from the iris, and then proceeding to the withdrawal of the ring (1) from the ocular globe, finalizing the surgery.

1- IRIS EXPANSION RING FOR CATARACT SURGERY WITHOUT INJECTOR a ring (1) predominantly circular-shaped with a higher diameter (2) in the center of its latitudinal section and higher height (3) in the center of its latitudinal section, displaying an opening (7) in its inferior section; characterizes by an opening (7) sided by hook-shaped support points (8) in each of its inferior extremities, one to five, preferably three inferior structures (5) for fitting to the iris edge, triangular-shaped and displaying an orifice (6) for ring manipulation, three to six, preferably four superior structures (4) for fitting on top of the iris, rectangular-shaped and displaying an orifice (6) for ring (1) manipulation. Those superior structures (4) alternate along the circumference with the inferior structures (5), which are smaller than the superior ones (4). The planes containing the superior structures are parallel among themselves, the same occurring with the inferior structures (5), also parallel among themselves; these parts are distanced to create a socket for iris accommodation; the internal diameter of the ring ranges from 5 to 8 mm, preferably 6.3 mm, and its height ranges from 0.2 to 1.1 mm, preferably 0.6 mm.

2- IRIS EXPANSION RING FOR CATARACT SURGERY WITHOUT INJECTOR according to vindication 1 since the implantation of the ring (1) can be carried on through the same incision made for withdrawal of the cataract, and insertion can start by any of the support points (8) of the ring (1); the ring (1) can be inserted in the ocular globe with the help of simple tweezers commonly used in cataract surgery; once inserted, one may use a hook used for manipulation of the cataract lens; this hook must be inserted in each orifice (6) fitting each of the structures, superior (4) and inferior (5), to patient's iris until its pupil is fully dilated by the ring; next, one can proceed with the surgery and after implantation of the intraocular lens, the lens manipulating hook is again used to detach each of the structures, superior (4) and inferior (5), from the iris, and then proceed with the removal of the ring (1) from the ocular globe and finalizing the surgery.
